(19) United States
(12) Patent Application Publication
(10) Pub. No.: US 2018/0000488 A1
(43) Pub. Date: Jan. 4, 2018

(54) INFLATABLE INTRAUTERINE BALLOON

(71) Applicant: JMD Innovation Inc., Burnaby (CA)

(72) Inventors: Lina HU, Burnaby (CA); Huafeng LI, Burnaby (CA); Ji ZHANG, Burnaby (CA)

(21) Appl. No.: 15/520,825

(22) PCT Filed: Oct. 22, 2015
(86) PCT No.: PCT/CA2015/051072

§ 371 Date: Apr. 20, 2017

Related U.S. Application Data
(60) Provisional application No. 62/067,030, filed on Oct. 22, 2014.

(51) Int. Cl. A61B 17/12; A61M 25/10; A61M 1/00; A61B 17/42; A61B 17/00

(52) U.S. Cl. CPC A61B 17/12136; A61M 1/008; A61B 17/42; A61M 25/10185; A61M 25/1002; A61B 2017/12004; A61B 2017/00862; A61M 1/0019; A61B 2017/4216; A61M 2025/105; A61M 2025/1061; A61M 2025/1075; A61M 2205/3344

(57) ABSTRACT

Disclosed herein are inflatable balloon apparatuses for use in stopping massive blood flow from a uterine wall due trauma or disease. Also disclosed is a method for using the apparatuses.

### CROSS-REFERENCE TO RELATED APPLICATIONS

This application claims the benefit of U.S. Provisional Application No. 62/067,030, filed Oct. 22, 2014, the contents of which are incorporated herein by reference in their entirety.

### TECHNICAL FIELD

The subject matter described herein relates to a uterine balloon for the purpose of mitigating and/or stopping blood flow from the uterine wall following trauma. The presently disclosed apparatus is introduced into a uterus which is experiencing massive blood flow then inflated to create pressure to stop blood flow. The devices described herein also provide for the flow of blood and blood clots from the uterus.

### BACKGROUND

Hemorrhage continues to be one of the major causes of maternal deaths generally, with obstetrical hemorrhage being the third leading cause of maternal death by hemorrhage in the United States. Worldwide, maternal hemorrhage qualifies as the leading cause of maternal death.

Techniques for managing obstetrical hemorrhage may be medical, mechanical, or surgical. Hysterectomy, while an effective surgical procedure for treating this condition, bears severe consequences, in particular for young women who have not completed childbearing.

One of the mechanical procedures often used for managing obstetrical hemorrhage involves packing the uterus with heavy gauze. This procedure remains controversial because of a high failure rate, and is considered a waste of time by some medical practitioners. The high failure rate is attributable to the inherent difficulty in packing the uterus properly so that there is an even distribution of pressure along the entire inner surface of the organ.

Accordingly, a more effective procedure is needed to address uterine hemorrhage and bleeding. Ideally, the procedure should present minimal long-term health consequences to the patient, be quickly and easily accomplished, be easily learned and require no specialized instrumentation.

Postpartum hemorrhage is an obstetrical emergency that can follow vaginal or cesarean delivery. It is one of the leading causes of maternal mortality, especially in the developing world. Early use of intrauterine balloon tamponade is a way of limiting ongoing uterine blood loss while initiating other measures, and can be readily implemented by providers with minimal training. It can be a life-saving intervention, especially in low-resource settings where blood transfusion and surgical facilities may not be available. Multiple types of balloons catheters are available for uterine tamponade.

Intrauterine balloon tamponade can be a relatively easy-to-use and effective second-line option for managing severe uterine hemorrhaging, but cost and other factors limit its use in low-resource settings. With further research and development, a lower-cost, appropriate intrauterine balloon tamponade device has the potential to vastly improve treatment for uterine hemorrhaging and save women's lives.

The precise mechanism of action for intrauterine balloon tamponade is still unclear. In the case of postpartum hemorrhaging, the placenta is a low-pressure system, so it seems likely that when the placenta is the source of hemorrhage, the direct pressure of the balloon, even well below systemic pressure, will halt bleeding. When the hemorrhage is instead from an arterial source in the endometrium, it is possible that the balloon's exerted pressure exceeds the arterial pressure and thus promotes clot formation. A third possibility is that the introduction of the balloon in the atonic uterus causes it to contract.

The foregoing examples of the related art and limitations related therewith are intended to be illustrative and not exclusive. Other limitations of the related art will become apparent to those of skill in the art upon a reading of the specification and a study of the drawings.

### BRIEF SUMMARY

The following aspects and embodiments thereof described and illustrated below are meant to be exemplary and illustrative, not limiting in scope.

The present disclosure is directed to an apparatus having an inflatable balloon comprising an inner and an outer layer, wherein the balloon shape, when inflated, takes on the shape of the site to be controlled (for example, the shape of a uterine wall). An insufflation tube feeds a distending medium (such as water or a physiologic fluid, or alternatively a gas) through a supply tube to inflate the balloon. The apparatus comprises a self-pressure control for maintaining a constant pressure inside the balloon as until the bleeding reduces significantly or stops. The inflated balloon presses against the uterine wall to check the flow of bleeding from the wall. One benefit of the balloon is that it is effective when bleeding occurs at multiple sites in the uterine wall.

The presence of the outer balloon layer allows clotted blood to be drained as needed.

The apparatus also comprises an internal drain tube coaxially and sealably positioned with respect to the balloon, and having a port at an inserted end for draining blood and other fluids from the uterine cavity.

In one embodiment, the self-pressure control may take on various forms, each of which produces a gradual discharge of fluid from the balloon to maintain a constant pressure inside of the balloon as necessary to control or stop bleeding. The control includes a series of valves according to devices known in the art. In one aspect of the disclosure, the control includes a combination fill and pressure regulating valve. In another aspect of the invention the control includes a combination fill and pressure relief valve. In still another aspect of the invention the control includes a high/low pressure warning device.

With sufficient inflation pressure, the balloon exerts a uniform distribution of compressive pressure, which in turn controls bleeding sites. This control of bleeding gives the uterus time to respond normally as bleeding stops, and conserves blood while preparing the patient for surgery as necessary. This device may also be utilized in the vagina to control bleeding associated with submucosal tears (tears under the mucosal vaginal lining), and with any continuous bleeding resulting in the subsequent development of hematomas. Accordingly, significant blood loss, pain, and the possibility of an operative procedure may be prevented. In a preferred embodiment of the present invention, an outer surface of the balloon is coated or impregnated with a hemostatic material for contact with the uterine wall or vaginal lining to provide additional control of bleeding.

An advantage provided by the present invention is that its insertion, inflation, and removal require no surgical procedures, and very little time. The device is thus uniquely and admirably suitable for use in uterine hemorrhaging emergencies. Other presently known devices which use expandable material to stop abdominal bleeding during or after surgery are considerably more complicated in construction and much more difficult to use, often requiring surgical application and removal.

In an alternative embodiment, the apparatus comprises a balloon having a single layer wherein the single layer comprises a tread structure which protrudes from the external surface of the balloon single layer so as to provide a space between the balloon single layer and the uterine wall.

Although the devices as disclosed herein may be used in various surgical procedures, its use will be described in detail only in connection with an improved cesarean section surgical procedure.

### BRIEF DESCRIPTION OF DRAWINGS

FIG. 1 is a diagram of a uterine balloon apparatus comprising an outer layer and an inner layer.

FIG. 2 illustrates embodiments of uterine balloon shape upon inflation.

FIG. 3 is a diagram of a uterine single layer balloon apparatus comprising a tread structure.

FIG. 4 is a diagram of a delivery device for delivery of a uterine balloon apparatus.

### DETAILED DESCRIPTION

Various aspects now will be described more fully hereinafter. Such aspects may, however, be embodied in many different forms and should not be construed as limited to the embodiments set forth herein; rather, these embodiments are provided so that this disclosure will be thorough and complete, and will fully convey its scope to those skilled in the art.

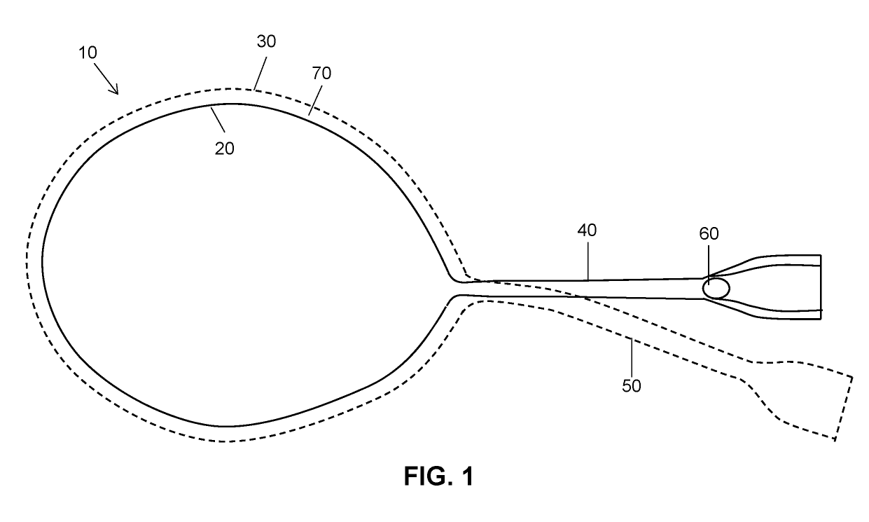

FIG. 1 illustrates a first embodiment of a uterine balloon apparatus 10, according to the present disclosure. In FIG. 1, an inflated balloon comprising an inner layer 20 and an outer layer 30 are shown in an inflated configuration. An outlet tube 50 extends from outer layer 30. A semi-rigid catheter 40 extends part way into the inflated balloon inner layer 20 and facilitates the insertion and removal of the balloon from the uterus. At an inner end portion of the catheter 40 there is a series of small apertures for infusing a physiologic fluid, such as a normal saline solution, into the balloon after it is inserted into the uterus.

In one embodiment, the balloon inner layer 20 and/or outer layer 30 is fabricated from an expandable material, including, but not limited to, polyurethane, silicone, or another medical-grade elastomeric material.

In one embodiment, the outlet tubing may comprise medical-grade nylon, polyurethane, polyethylene, polyvinyl chloride, silicon, natural rubber, synthetic rubber, or other suitable material.

The portion of the catheter 40 which is outside of the uterus is connected to a control system which is comprised of, for example, a pressure relief valve, a pressure gauge, a combination fill and pressure regulator valve 60 and short lengths of flexible tubing.

The primary purpose of the control system is to maintain a constant fluid pressure inside of the balloon inner layer 20. Another purpose to prevent the pressure inside of the balloon inner layer 20 from exceeding a level which could cause premature ejection of the balloon or injure the uterus. In a normal operating mode, a desired pressure which is less than a maximum allowable pressure, is maintained constant by gradually discharging some of the physiologic fluid from the balloon inner layer 20 if necessary in order to allow the uterus to resume its normal physiologic function.

Inflation of apparatus 10 as shown in FIG. 1 results in the formation of a chamber between inner layer 20 and outer layer 30 of the balloon. Blood from the uterine wall can flow into this chamber, some of which will form clots. Blood and blood clots are then able to pass through outlet tube 50. In one embodiment, a device outside of the uterus may collect and measure the output of blood from the uterus.

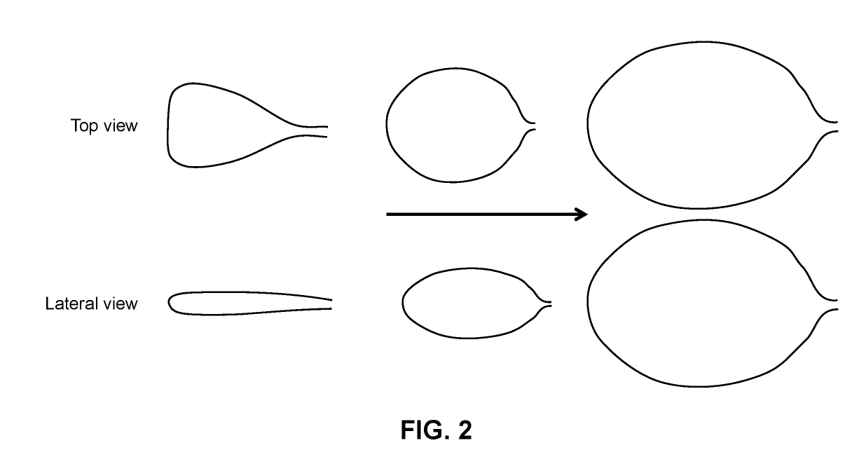

FIG. 2 provides a depiction of various shapes which may be taken by the balloon apparatus as it inflates within the uterus. FIG. 2 provides both a top and a lateral view. Importantly, the balloon inflates to match the size and shape of the uterus. In one embodiment a first and second plane of maximum diameter can be located in any position that allows the balloon to have a substantially pear-shaped appearance. For example is a non-limiting embodiment where the first plane of maximum diameter is located distally with respect to a longitudinal midpoint of the balloon. In an alternative non-limiting embodiment, the second plane of maximum diameter is located proximally on the catheter body with respect to the balloon's longitudinal midpoint.

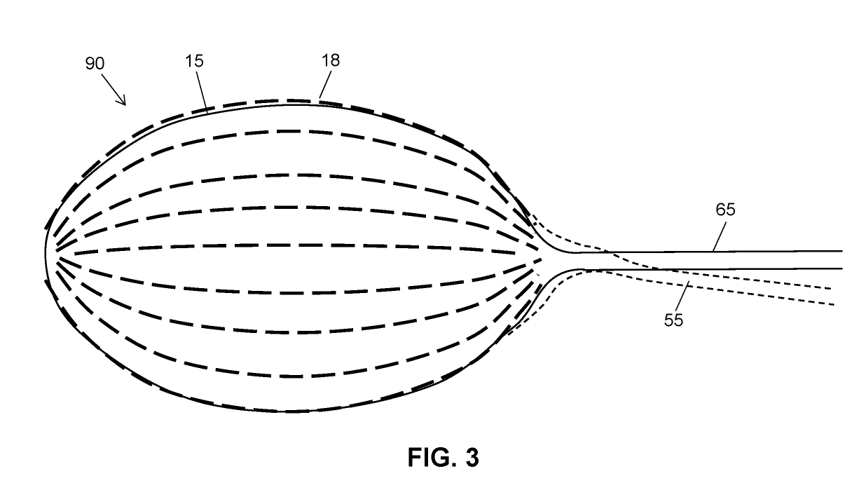

In FIG. 3, a second embodiment 90 is shown, wherein the apparatus comprises a single layer balloon 15 which includes a tread structure 18. As a result, a space is created between single layer balloon 15 when inflated and the uterine wall, forming a chamber similar to that shown in FIG. 1 where a chamber is formed between inner layer 20 and outer layer 30. As shown in FIG. 3, a negative pressure can form to facilitate removal of any blood clots through an output tube 55.

FIG. 3 shows that the single layer balloon 15 of balloon apparatus 90 comprises a semi-rigid catheter 65 similar to that shown as semi-rigid catheter 40 shown in FIG. 1 for apparatus 10. Catheter 65 extends part way into the inflated single layer balloon 15 and facilitates the insertion and removal of balloon apparatus 90 from the uterus. At an inner end portion of the catheter 90 there is a series of small apertures for infusing a physiologic fluid, such as a normal saline solution, into the balloon after it is inserted into the uterus. In one embodiment, single layer 15 is fabricated from an expandable material, including, but not limited to, polyurethane, silicone, or another medical-grade elastomeric material.

The portion of the catheter 65 which is outside of the uterus is connected to a control system which is comprised of, for example, a pressure relief valve, a pressure gauge, a combination fill and pressure regulator valve and short lengths of flexible tubing.

As with apparatus 10, the primary purpose of the control system for balloon apparatus 90 is to maintain a constant fluid pressure inside of single layer balloon 15. Another purpose to prevent the pressure inside of single layer balloon from exceeding a level which could cause premature ejection of the balloon or injure the uterus. In a normal operating mode, a desired pressure which is less than a maximum allowable pressure is maintained constant by gradually discharging some of the physiologic fluid from single layer balloon if necessary in order to allow the uterus to resume its normal physiologic function.

In one embodiment, a pressure control unit is present at the proximal end of each of balloon apparatuses 10 and 90. The pressure control unit is configured as is readily apparent to the ordinarily skilled artisan. As a non-limiting example, a pressure relief valve is located close to the uterus to assure that the pressure in the balloon will not exceed the maximum limit under all conditions, such as pinched tube or a mal functioning pressure regulator valve. Numerous known types of relief valves, such as a ball check valve are available for preventing the pressure from exceeding the maximum limit. A generally cylindrical shaped valve is comprised of a thin resilient wall housing having a pair of flat walls which abut each other to seal the valve when the pressure is below the maximum limit separate and release fluid when the pressure is at the maximum limit. The thickness and diameter of the walls allow the walls to separate and open the valve along the horizontal axis and discharge fluid when the pressure is at the maximum limit.

A pressure gauge in one embodiment consists of an existing type of transducer and an existing type of analog or digital meter. The meter is positioned near a patient, to enable a nurse or a physician to monitor the pressure in the balloon when and after liquid is infused but may be or at a remote nurses station for monitoring the pressure. In one embodiment, the valve is a ball check valve having an inlet housing, an outlet housing, a spherical ball and a spring. The outlet housing threadably engages the inlet housing with preferably a sealing type thread, such as a dryseal thread. One unique feature of the valve is that an exterior portion of the outlet housing thread is removed and marked with graduations to indicate the relative positions of the inlet and outlet housings. The relative position of the inlet housing and the outlet housing is adjusted by rotating the outlet housing relative to the inlet housing.

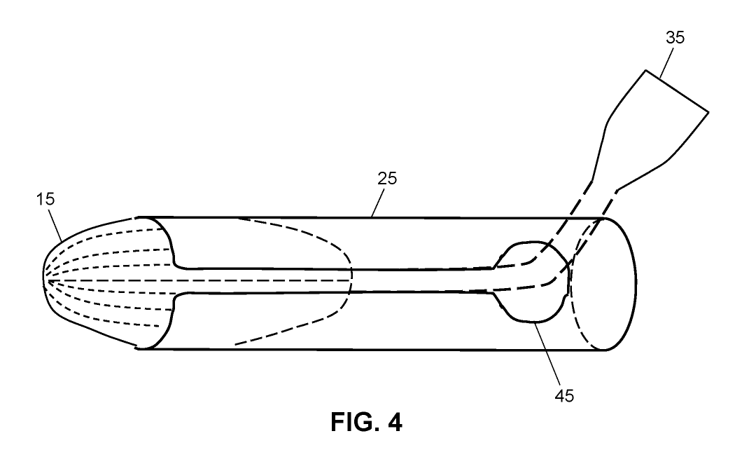

Another aspect of balloon apparatuses 10 and 90 is a delivery device is illustrated in FIG. 4. Here, a tubular sheath 25 encases the balloon apparatus in an uninflated configuration prior to delivery to the uterus. In one embodiment, tubular sheath 25 comprises a hole in the wall near the proximal end of tubular sheath 25 (depicted as 45 in FIG. 4).

The described device is used in the following manner. An empty, deflated balloon 15 is inserted into a bleeding uterus with the assistance of the catheter 25. A warm physiologic solution from an external source is infused into the balloon 15 through the distal end of the outlet tube 35. During the infusion of fluid, the balloon 15 distends and its effect on bleeding is observed. The pressure gauge may also assist in determining when stop the infusion of the fluid into balloon 15. The preferred pressure in balloon 15 is the lowest pressure which counters the bleeding. When this condition occurs, the outlet housing is rotated to set the pressure by cutting off further fluid infusion. The external source of the fluid is disconnected and a drainage bag is attached to the outlet tube 35 to capture fluid which is discharged from balloon 15 to maintain the constant pressure. If the pressure should exceed the maximum allowable pressure, the pressure relief valve opens to prevent the pressure from exceeding the maximum allowable pressure. If periodic contractions of the uterus cause the pressure to rise, a ball within the control valve is lifted off its seat and fluid is gradually released to maintain the desired pressure. In this case, for example, after the uterus has relaxed, and the pressure in balloon 15 is at the set level, the ball returns to its seat and the pressure regulating valve is closed, preventing a further discharge of fluid. Thus, pressure regulator valve (shown as 60 in FIG. 1) maintains a constant pressure in balloon 15. If the system should malfunction because of a pinched tube or a faulty regulator valve, a pressure relief valve opens to prevent a premature expulsion of the balloon 15 or a rupture of the uterus.

From the foregoing it will be understood that my invention provides an effective means for terminating a moderate or massive flow of blood in a uterus following trauma or childbirth and is an alternative to major surgery, hysterectomies and massive blood transfusions for terminating hemorrhaging in a uterus.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. An inflatable balloon apparatus for treating obstetrical hemorrhaging, the apparatus comprising:
an inflatable balloon comprising an inner layer and an outer layer, the inner layer being inflatable to an inflated configuration to take on a shape of an obstetrical site to be controlled, the outer layer being positionable against a wall of the obstetrical site to control flow of blood from the wall, wherein in the inflated configuration, the balloon comprises a chamber between the inner layer and the outer layer for receiving flow of blood from the obstetrical site;
an insufflation tube coupled to the inner layer of the balloon for communication of a distending medium to inflate the balloon; and
an internal drain tube comprising an inserted end and a port at the inserted end, the drain tube being coupled to the chamber of the balloon, the drain tube being configured for draining blood or other fluids from the obstetrical site via the chamber of the balloon.

13. The apparatus of claim 12, wherein the insufflation tube is coaxially disposed within the drain tube at a proximal end of the balloon.

14. The apparatus of claim 12, wherein the insufflation tube comprises a semi-rigid catheter extending into the balloon inner layer, the insufflation tube facilitating insertion and removal of the balloon from the obstetrical site.

15. The apparatus of claim 14, wherein the catheter comprises an inner end portion and a series of apertures at the inner end portion for infusing a physiologic fluid into the balloon after insertion of the balloon into the obstetrical site.

16. The apparatus of claim 12, further comprising a pressure control unit in communication with the inner layer for maintaining a constant pressure inside the balloon as until bleeding reduces significantly or stops.

17. The apparatus of claim 16, wherein the pressure control unit comprises a series of valves to produce a gradual discharge of fluid from the balloon to maintain a constant pressure inside of the balloon for controlling or stopping bleeding.

18. The apparatus of claim 12, wherein an outer surface of the balloon is coated with a hemostatic material for contact with the wall of the obstetrical site to provide additional control of bleeding.

19. The apparatus of claim 12, wherein an outer surface of the balloon is impregnated with a hemostatic material for contact with the wall of the obstetrical site to provide additional control of bleeding.

20. The apparatus of claim 12, further comprising a pressure gauge for monitoring pressure of the balloon.

21. A balloon apparatus for treating obstetrical hemorrhaging, the apparatus comprising:
an inflatable balloon configured to be inserted into an obstetrical site, the balloon having a single layer, an external surface, and a tread structure formed on the external surface of the balloon, the tread structure defining innermost and outermost surfaces and a chamber collectively formed by grooves of the tread structure, the tread structure protruding from the external surface, the tread structure defining a chamber of the balloon to provide a space between the single layer and a wall of the obstetrical site;
an insufflation tube coupled to the balloon for communication of a distending medium to inflate the balloon; and
a drain tube coupled to the chamber of the balloon for draining blood or other fluids from the obstetrical site via the chamber of the balloon.

22. The apparatus of claim 21, wherein the chamber is formed when the balloon is inflated.

23. The apparatus of claim 21, wherein the insufflation tube is coaxially disposed within the drain tube at a proximal end of the balloon.

24. The apparatus of claim 21, further comprising a semi-rigid catheter coupled to the balloon and extending into at least partially into the balloon to facilitate insertion and removal of balloon apparatus from the obstetrical site.

25. The apparatus of claim 21, wherein a proximal portion of the catheter, configured to be positioned outside of the obstetrical site, is connected to a pressure control unit.

26. The apparatus of claim 21, further comprising a pressure control unit coupled to a proximal end of the balloon apparatus, the pressure control unit being configured to control pressure within the balloon to prevent the pressure from exceeding a maximum allowable pressure.

27. A method for treating obstetrical hemorrhaging, the method comprising:
inserting an inflatable balloon into an obstetrical site, the balloon comprising an inner layer and an outer layer;
inflating the inner layer via an insufflation tube coupled to the inner layer until the outer layer is positioned against and takes on a shape of the obstetrical site;
permitting flow of blood from the obstetrical site along the outer layer via an internal drain tube fluidly coupled to the outer layer; and
draining blood and other fluids from the obstetrical site via a port at an inserted end of the drain tube.

28. The method of claim 27, further comprising maintaining a constant fluid pressure inside of the balloon inner layer.

29. The method of claim 27, further comprising limiting pressure inside the balloon inner layer from exceeding a maximum allowable pressure which could cause premature ejection of the balloon or injure the obstetrical site.

30. The method of claim 29, wherein the limiting pressure comprises maintaining a desired pressure, less than the maximum allowable pressure, by gradually discharging a distending medium from the balloon inner layer.

31. The method of claim 27, further comprising applying a negative pressure to facilitate removal of any blood clots through the drain tube.
